(19) United States
(12) Patent Application Publication
(10) Pub. No.: US 2017/0007646 A1
(43) Pub. Date: Jan. 12, 2017

(54) METHODS OF MANIPULATING ALGINATE MICROCAPSULE SIZE AND PERMEABILITY

(71) Applicant: The Regents of the University of California, Oakland, CA (US)

(72) Inventors: Jonathan R.T. Lakey, Irvine, CA (US); Rahul Krishnan, Santa Ana, CA (US); Elliot L. Botvinick, Carlsbad, CA (US)

(21) Appl. No.: 15/204,916

(22) Filed: Jul. 7, 2016

Related U.S. Application Data
(60) Provisional application No. 62/189,983, filed on Jul. 8, 2015.

(51) Int. Cl. A61K 35/39; A61K 9/00; A61K 9/48; A61K 9/50; A61J 3/00

(52) U.S. Cl. CPC A61K 35/39; A61K 9/5036; A61J 3/00; A61K 9/4833; A61K 9/0024

(57) ABSTRACT

Methods and apparatuses are disclosed for the treatment of diabetes using artificial islets of Langerhans. In one example, the artificial islet of Langerhans include islets or stem cells (but can also include hepatocytes or even any biological cell type) encapsulated in alginate microcapsules. The microcapsules can then be shrunk to reduce dead space between the capsules and the cells by incubating at physiological human temperatures and/or alginate crosslinking in the presence of barium chloride.

### PRIORITY APPLICATIONS

This application claims the benefit of U.S. Provisional Patent Application No. 62/189,983, filed Jul. 8, 2015. The entire disclosure of all of these priority documents is hereby incorporated by reference in its entirety.

### BACKGROUND

Field

The present disclosure relates to systems and methods for the treatment of diabetes, and more particularly to a system and method for the reduction of the diffusion distance between the outer surface of a bioencapsulation device and the surface of biological cells (e.g. islets of Langerhans or stem cells) encapsulated therein.

Description of the Related Art

Diabetes is the 4th leading cause of death in the United States with more than 3 million Americans currently suffering from Type 1 diabetes (“T1D”). A promising treatment for T1D is the transplantation of donor islet or stem cells to restore euglycemia (e.g. a normal level of sugar in the blood). However, clinical islet transplantation currently requires a lifetime of immune-suppression therapy and is encouraged only for diabetic patients with life-threatening complications. Therefore, encapsulation of islet or stem cells is a promising strategy that prevents direct contact between implanted islet or stem cells and the host's immune system that may allow patients to receive islet transplants without requiring lifelong immunosuppressive therapy.

Encapsulation of islet or stem cells within permeable hydrogels provides a partial immune barrier and reduces the need for pharmacological immunosuppression for allografts. Advances in encapsulation technology have augmented islet viability (>80%) and graft function while reducing the minimal required curative islet dose.

However, islet or stem cell encapsulation negatively impacts islet or stem cell function due to a ‘dead space’ consisting of biopolymer coating that is necessary in order to protect the encapsulated cells from attack by the body's immune system. In the native location within the pancreas, the islets of Langerhans are provided with a rich network of blood vessels that ensure adequate nutrient and oxygen supply as this tissue is highly metabolically active. However, in the case of bioencapsulated islet or stem cells, the ‘dead space’ that surrounds the islet or stem cells can greatly reduce oxygen and nutrient bioavailability due to the fact that oxygen and other vital nutrients will need to diffuse a certain distance in order to reach the islet or stem cells. This diffusion is passive as it is driven solely by a concentration gradient.

### SUMMARY

Embodiments of the present disclosure are related to bioencapsulation devices and methods for optimizing the diffusion distance between the bioencapsulation device and a tissue surface (e.g. the islets of Langerhans).

Disclosed is method to achieve a significant reduction in the diffusion distance between the outer surface of the bioencapsulation device (e.g. alginate microcapsules) and the surface of the islets or stem cells. In some embodiments, crosslinking alginate microcapsules at temperatures close to physiological body temperatures (37°±5° C.) can result in a significant, irreversible reduction in microcapsule size, volume and pore size. In some embodiments, incubating alginate microcapsules with barium chloride can result in a significant, irreversible reduction in microcapsule size, volume, and pore size. In some embodiments, generating alginate microcapsules with high guluronate alginate can result in a significant, reduction in microcapsule size, volume, and pore size. In some embodiments, the reduction in microcapsule size, volume, and pore size is irreversible.

### BRIEF DESCRIPTION OF THE DRAWINGS

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure.

FIG. 1 illustrates a graph showing the effect of barium on sodium-induced swelling in ultra-pure low viscosity D-mannuronate (“UP LVM”) microcapsules. Similar results have been obtained with ultra-pure low viscosity D-guluronate (“UP LVG”) microcapsules.

FIGS. 2A-2E illustrates the effect of in vitro incubation at various temperatures on alginate microcapsule diameter using 2.5% UP LVM over different time periods.

FIGS. 3A-3E illustrate the percentage decrease in volume after various time points with different temperature incubations using 2.5% UP LVM.

FIG. 4 illustrates Dextran permeability studies using green fluorescent 150 kDa Dextrans at different temperatures.

FIG. 5A illustrates the dextran diffusion assessment in 2.5% UP LVG Alginate Microcapsules incubated at 3° C. on Day 7 over a period of time.

FIG. 5B illustrates the dextran diffusion assessment in 2.5% UP LVG Alginate Microcapsules incubated at 37° C. on Day 7 over a period of time.

FIG. 5C illustrates the dextran diffusion assessment in 2.5% UP LVM Alginate Microcapsules incubated at 3° C. on Day 7 over a period of time.

FIG. 5D illustrates the dextran diffusion assessment in 2.5% UP LVM Alginate Microcapsules incubated at 37° C. on Day 7 over a period of time.

FIG. 6A illustrates the dextra diffusion assessment in 2.5% UP LVG and 2.5% UP LVM Alginate Microcapsules Incubated at 3° C. on Day 0 over a period of time.

FIG. 6B illustrates the dextra diffusion assessment in 2.5% UP LVG and 2.5% UP LVM Alginate Microcapsules Incubated at 37° C. on Day 1 over a period of time.

FIG. 6C illustrates the dextra diffusion assessment in 2.5% UP LVG and 2.5% UP LVM Alginate Microcapsules Incubated at 3° C. on Day 7 over a period of time.

FIG. 6D illustrates the dextra diffusion assessment in 2.5% UP LVG and 2.5% UP LVM Alginate Microcapsules Incubated at 37° C. on Day 7 over a period of time.

FIG. 6E illustrates the dextra diffusion assessment in 2.5% UP LVG and 2.5% UP LVM Alginate Microcapsules Incubated at 3° C. on Day 14 over a period of time.

FIG. 6F illustrates the dextra diffusion assessment in 2.5% UP LVG and 2.5% UP LVM Alginate Microcapsules Incubated at 37° C. on Day 14 over a period of time.

FIGS. 7A-7B illustrates the effect of in vitro incubation at various temperatures on alginate microcapsule diameter using 1.5% UP LVM over different time periods.

FIG. 8 illustrates the percentage decrease in volume after various time points with different temperature incubations using 1.5% UP LVM.

### DETAILED DESCRIPTION

Since diffusion is a function that is inversely proportional to the square of the distance between the encapsulated islet or stem cells and the vasculature immediately outside the bioencapsulation device, it stands to reason that any reduction in the ‘dead space’ would theoretically improve tissue oxygenation within cells including islets or stem cells. However, no technology currently exists to safely and effectively achieve this.

Disclosed is a simple, consistent and effective technique to reduce alginate microcapsule diameter and pore size after encapsulation and prevent sodium-induced microcapsule swelling. This can positively impact islet or stem cell function and consequently, improve transplant success rates. These changes can be of significance because a reduction in diameter can both have a positive impact on islet or stem cell function and survival as it reduces the diffusion distance, thereby improving islet or stem cell oxygenation. The reduction in pore size can also positively impact transplant outcomes as smaller pores can show greater exclusion of antibodies and immune cells when compared to alginate capsules with larger pore sizes. In some examples, alginate pore size can be a crucial parameter that aids in the protection of islets from host immune recognition, while allowing insulin, oxygen, and other micronutrients to diffuse through the capsules. Islet encapsulation within alginate hydrogels can also be advantageous because it prevents direct contact between the encapsulated islets and the host immune system while significantly reducing the need for chronic systemic immunosuppression.

In some embodiments, disclosed is a method for reducing the volume of alginate microcapsules, comprising incubating the alginate microcapsules at 37°±5° C. under conditions sufficient to reduce the microcapsule volume.

In some embodiments, disclosed is a method for producing microcapsules comprising alginate encapsulated islet or stem cells. In some embodiments, the method comprises mixing islets or stem cells with alginate to form an islet or stem cell-alginate mixture, dripping the alginate-islet or stem cell mixture into a gelling solution to form alginate microcapsules, and incubating the microcapsules at 37°±5° C. for a time sufficient to shrink the alginate microcapsules thereby reducing the dead space between the alginate microcapsule and the encapsulated islet or stem cells.

In other embodiments, the method can further include a gelling solution comprising 10 mM-150 mM calcium chloride. In other embodiments, the method can further include the step of washing the alginate microcapsules with CaCl₂.

In other embodiments, the alginate microcapsules comprise a diameter which is reduced by 8-10% compared to alginate microcapsules not incubated at 37°±5° C. In other embodiments, the alginate microcapsules comprise a volume which is decreased by 20-30% compared to alginate microcapsules not incubated at 37°±5° C. In other embodiments, the alginate microcapsules comprising reduced pore size compared to alginate microcapsules not incubated at 37°±5° C.

In some embodiments, disclosed is a method for reducing the volume of alginate microcapsules. In some embodiments, the method comprises crosslinking the alginate microcapsules with barium chloride and incubating the alginate microcapsules at 37°±5° C. under conditions sufficient to reduce the microcapsule volume.

In some embodiments, disclosed is a method for producing microcapsules comprising alginate encapsulated islet or stem cells. In some embodiments, the method includes mixing islet or stem cells with alginate to form an alginate-islet or stem cell mixture, crosslinking the microcapsules with barium chloride by dripping the alginate-islet or stem cell mixture into a gelling solution composed of barium chloride, and incubating the microcapsules at 37°±5° C. for a time sufficient to shrink the alginate microcapsules thereby reducing the dead space between the alginate microcapsule and the encapsulated islet or stem cells.

In other embodiments, the method can further include a gelling solution of barium chloride is composed of between 10-150 mM of barium chloride. In other embodiments, the method can further include the step of washing the microcapsules with BaCl₂.

In other embodiments, the alginate microcapsules comprise a diameter which is reduced by 15-20% compared to alginate microcapsules not crosslinked with barium chloride. In other embodiments, the alginate microcapsules comprise a volume which is decreased by up to 40% compared to alginate microcapsules not crosslinked with barium chloride.

In other embodiments, the alginate used in any of the aforementioned embodiments can comprise 0.5-5.0 mL of 0.5%-5% ultra-pure low viscosity D-mannuronate (or D-Guluronate) alginate.

In some embodiments, disclosed is an apparatus for artificial islet or stem cell transplantation for the treatment of diabetes. In some embodiments, the apparatus comprises an alginate microcapsule comprising an alginate-islet or stem cell mixture, wherein the alginate-islet or stem cell mixture is treated with calcium chloride, and wherein the alginate microcapsule is incubated at 37°±5° C.

In other embodiments, the alginate microcapsules can comprise a diameter which is reduced by 8-12% compared to alginate microcapsules not incubated at 37°±5° C. In other embodiments, the alginate microcapsules can comprise a diameter which is reduced by 20-27% compared to alginate microcapsules not incubated at 37°±5° C.

In some embodiments, disclosed is an apparatus for artificial islet or stem cell transplantation for the treatment of diabetes. In some embodiments, disclosed is an alginate microcapsule comprising an alginate-islet or stem cell mixture wherein the alginate-islet or stem cell mixture is treated with barium chloride, and wherein the alginate microcapsule is incubated at 37°±5° C.

In other embodiments, the alginate microcapsules comprise a diameter which is reduced by up to 15-20% compared to alginate microcapsules not crosslinked with barium chloride. In other embodiments, the alginate microcapsules comprise a volume which is decreased by up to 40% compared to alginate microcapsules not crosslinked with barium chloride.

Shrinking Alginate Microcapsules by Crosslinking with Barium Chloride

In some embodiments, microcapsule size, volume, and pore size are reduced by crosslinking alginate microcapsules with barium chloride at temperatures close to physiological body temperatures (37°±5° C.).

### Microcapsule Assembly

Islet or stem cell preparations are first divided into aliquots ranging from 10 to 20,000 IEQ and centrifuged at 800×g for 2 minutes at 4° C., to facilitate the formation of a compact tissue pellet. The islet or stem cells are then gently mixed in 0.5-5 mL of 0.5%-5% ultra-pure low viscosity D-mannuronate (“UP LVM”) or D-guluronate (“UP LVG”) alginate (NovaMatrix, Norway). The alginate-islet or stem cell mixture is transferred into a glass syringe, fitted with a steel 25-28 gauge needle produced by Staedler Mars GmbH & Co. (Nurnberg, Germany) and mounted on an air-driven electrostatic microcapsule generator produced by Nisco Engineering Inc. (Oslo, Norway). The alginate-islet or stem cell mixture is allowed to drip from a height of 10-40 mm into a gelling solution composed of 10-150 mM sterile Barium Chloride which is gently and continuously agitated using a magnetic stirrer. The air pressure and voltage settings are maintained between 2-5 pounds per square inch (psi) and 9 kV respectively in order to generate consistently circular microcapsules with a mean diameter of 300 μm. The alginate microcapsules polymerized into circular bead-like structures with the islet or stem cells protected within.

### In Vitro Incubation

The encapsulated islet or stem cells are then washed with 5 mm BaCl₂ and then incubated in islet maturation media (UC Irvine) at 37°±5° C., 5% CO₂ in a standard incubator for 15-18 hours. At the end of this incubation period, a 15% reduction in microcapsule diameter and up to a 40% decrease in volume was noted. Compared to capsules gelled in Barium, capsules gelled in Calcium showed a 5-10% increase in diameter.

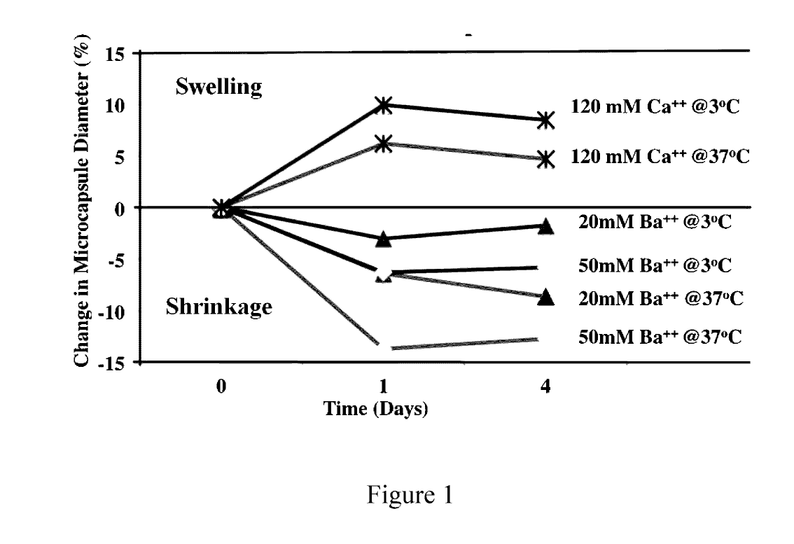

FIG. 1 illustrates the effect of Barium on sodium-induced swelling in UP LVM microcapsules. As illustrated, compared to capsules gelled in Calcium (*), those gelled in Barium (Δ, ♦) showed significant shrinkage at 37°±5° C. This shrinkage was synergistic with capsule shrinkage seen with incubation at physiological temperatures. Similar results have been obtained with ultra-pure low viscosity D-guluronate (“UP LVG”) microcapsules.

### Shrinking Alginate Microcapsules by Incubating at Certain Temperatures

In some embodiments, microcapsule size, volume, and pore size are reduced by incubating alginate microcapsules at temperatures close to physiological body temperatures (37°±5° C.).

### Microcapsule Assembly

Islet or stem cell preparations are first divided into aliquots of 10-20,000 IEqs and centrifuged at 800×g for 2 minutes at 4° C., to facilitate the formation of a compact tissue pellet. The islet or stem cells are then gently mixed in 0.5-1 mL of 0.5%-5% ultra-pure low viscosity D-mannuronate (“UP LVM”) or D-guluronate (“UP LVG”) alginate (NovaMatrix, Norway). The alginate-islet or stem cell mixture is transferred into a 2 mL glass syringe, fitted with a steel 25-28 gauge needle produced by Staedler Mars GmbH & Co. (Nurnberg, Germany) and mounted on an air-driven electrostatic microcapsule generator produced by Nisco Engineering Inc. (Oslo, Norway). The alginate-islet or stem cell mixture is allowed to drip from a height of between 10-40 mm into a gelling solution composed of 10-150 mM sterile Barium Chloride which is gently and continuously agitated using a magnetic stirrer. The air pressure and voltage settings are maintained at between 2-5 pounds per square inch (psi) and 9 kV respectively in order to generate consistently circular microcapsules with a mean diameter of 300 μm. The alginate microcapsules polymerized into circular bead-like structures with the islet or stem cells protected within.

### In Vitro Incubation

The encapsulated islets (or stem cells) can then washed with 5 mm BaCl₂ and then incubated in islet maturation media (UC Irvine) at 37°±5° C., 5% CO₂ in a standard incubator for 15-18 hours. At the end of this incubation period, an 8-10% reduction in microcapsule diameter and a 20-27% decrease in volume are noted.

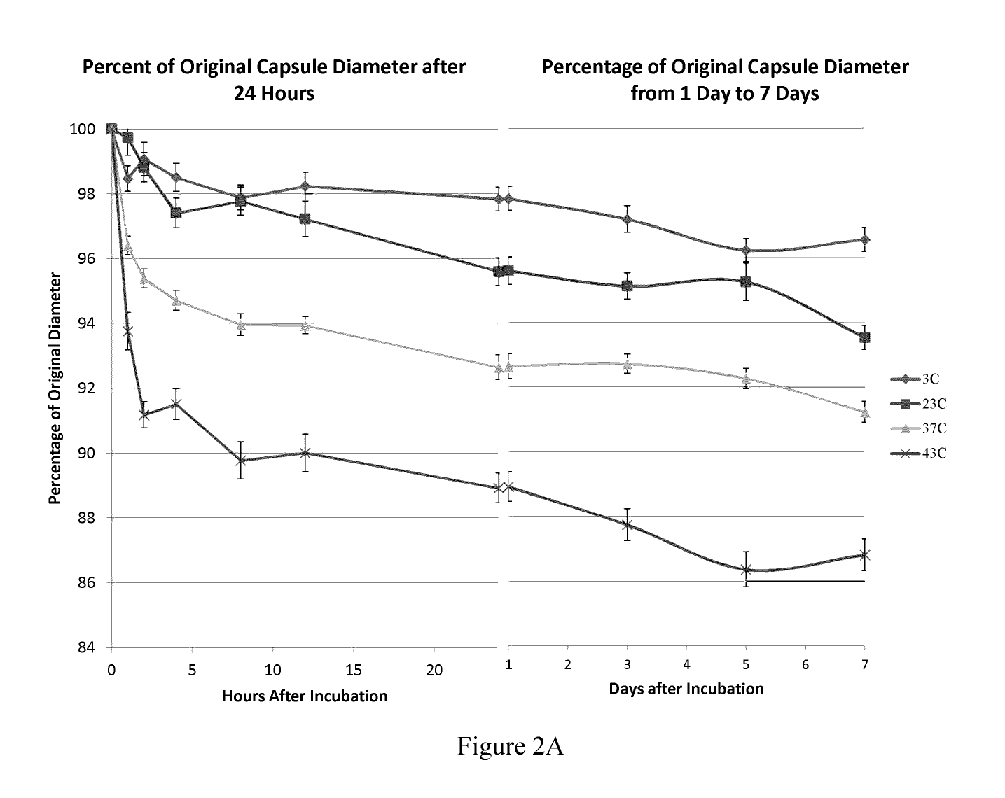

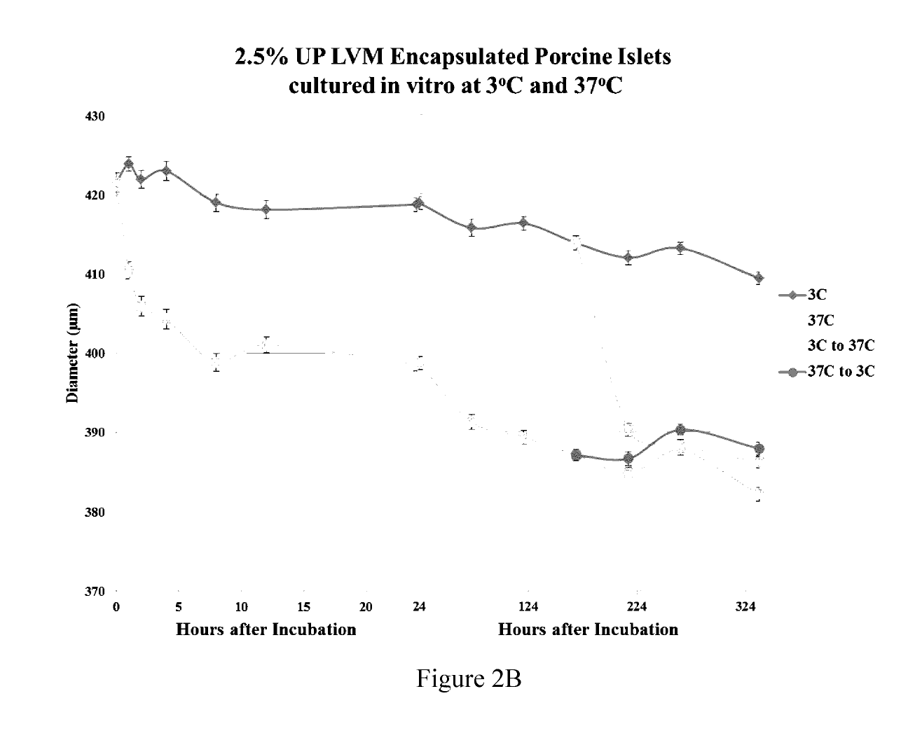

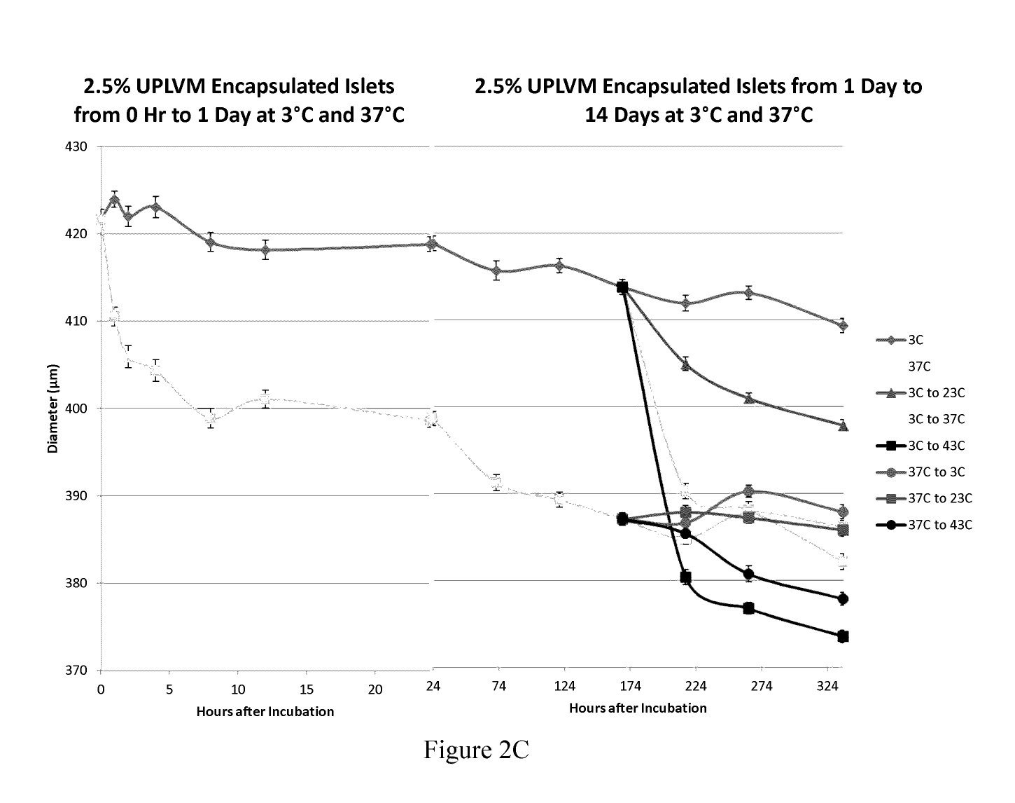

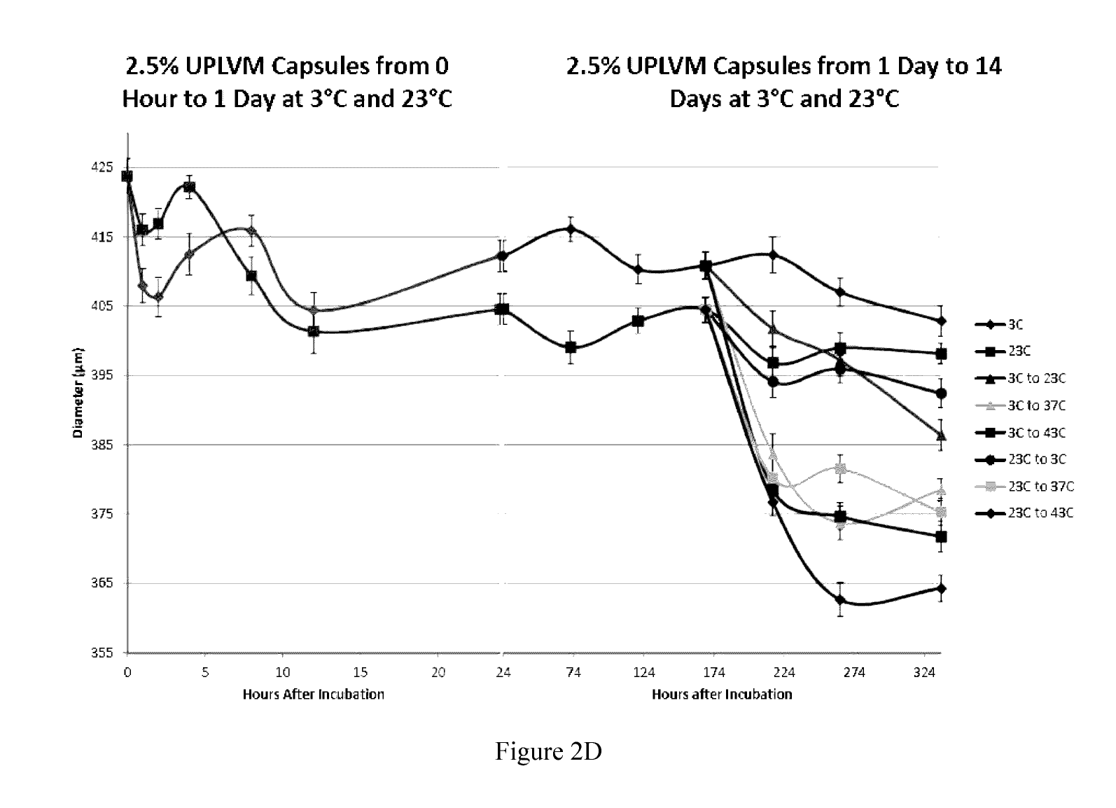

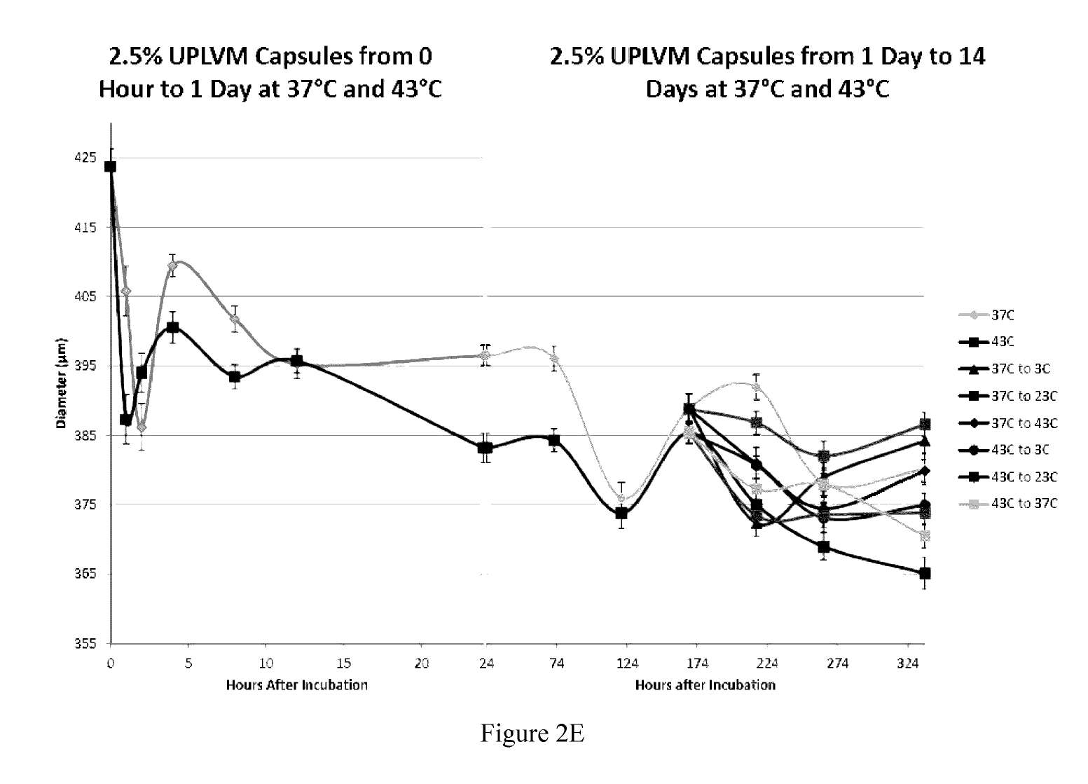

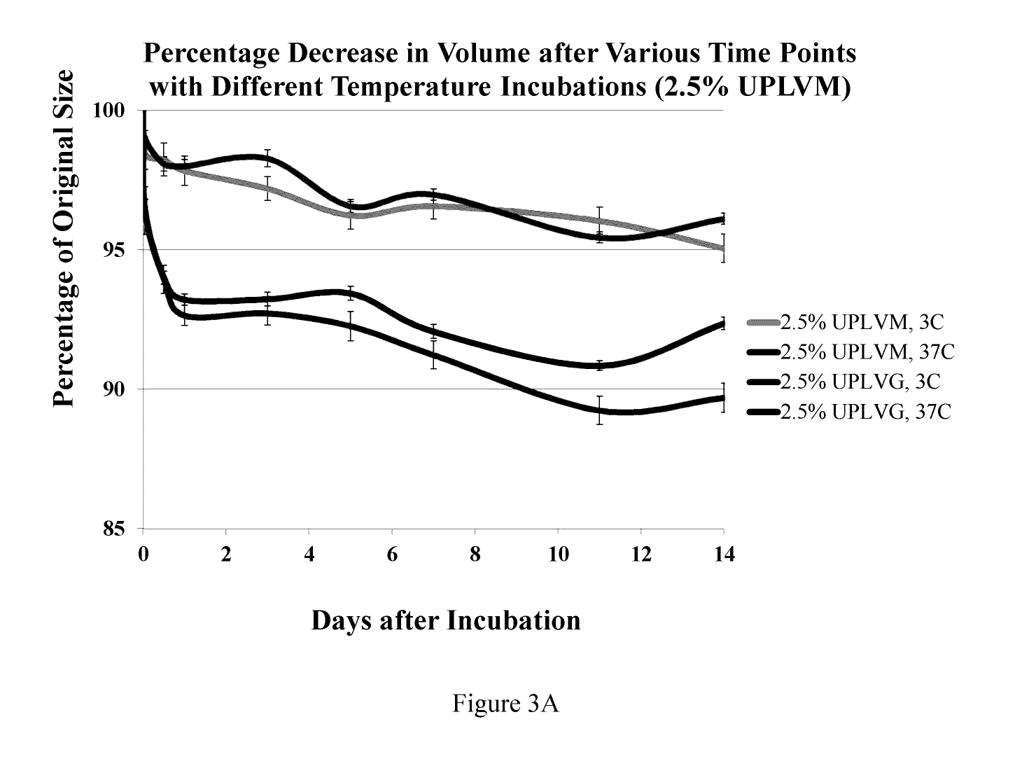

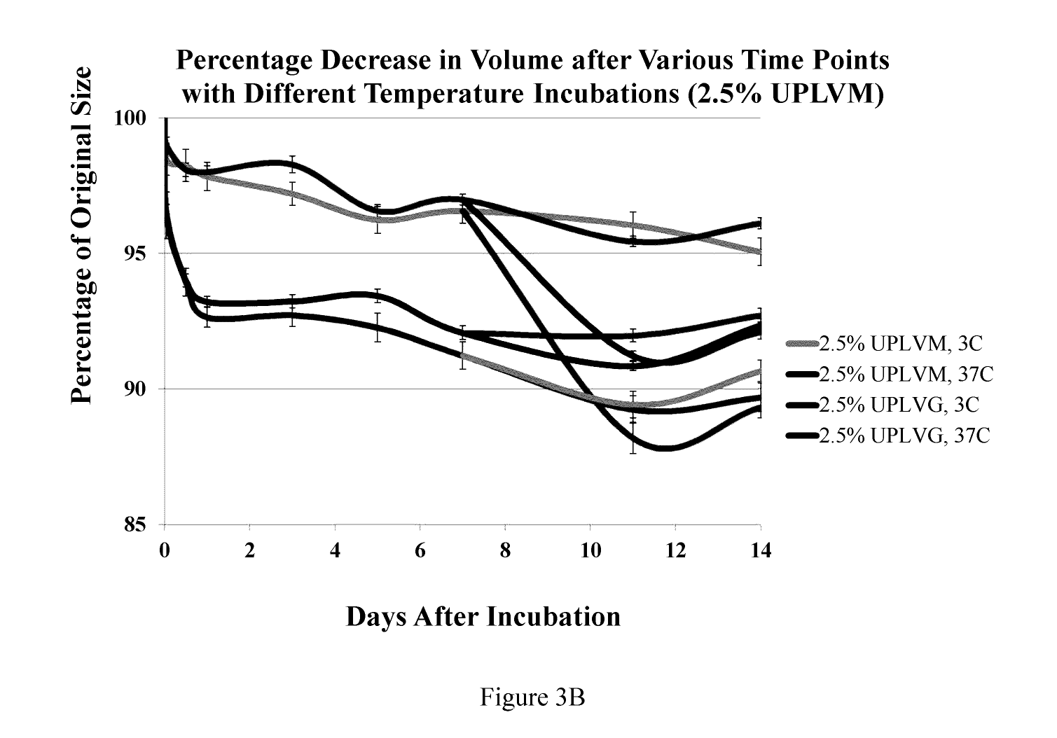

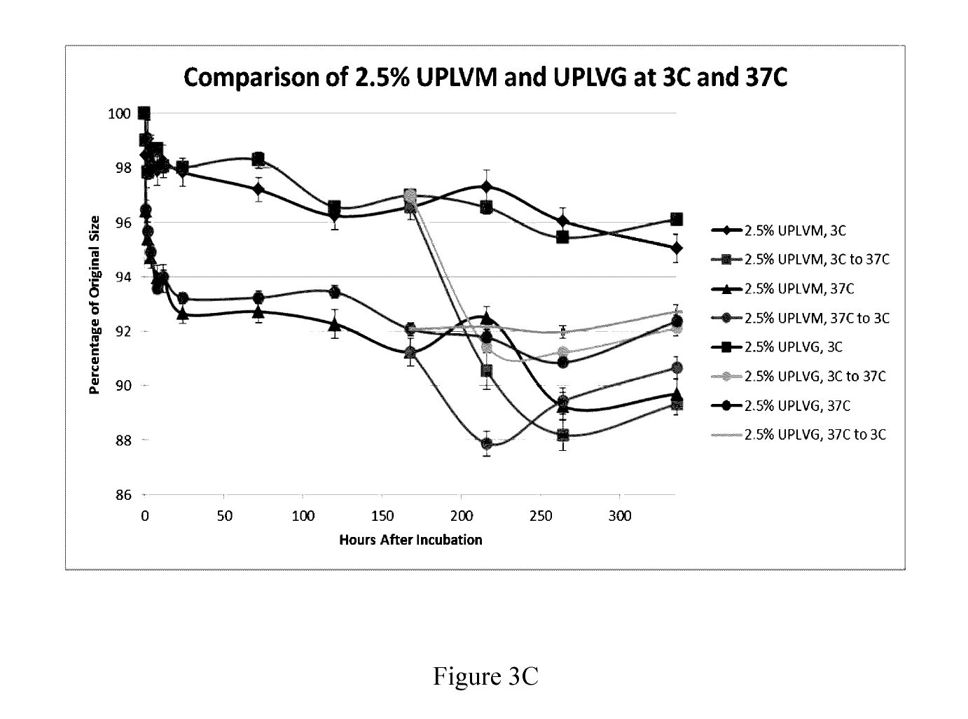

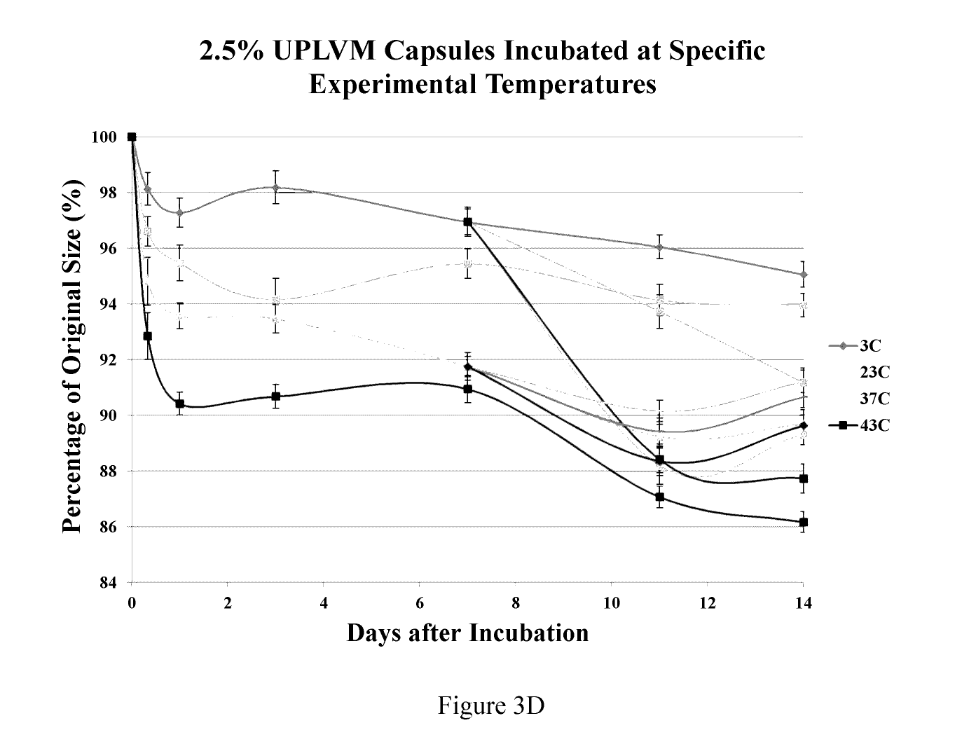

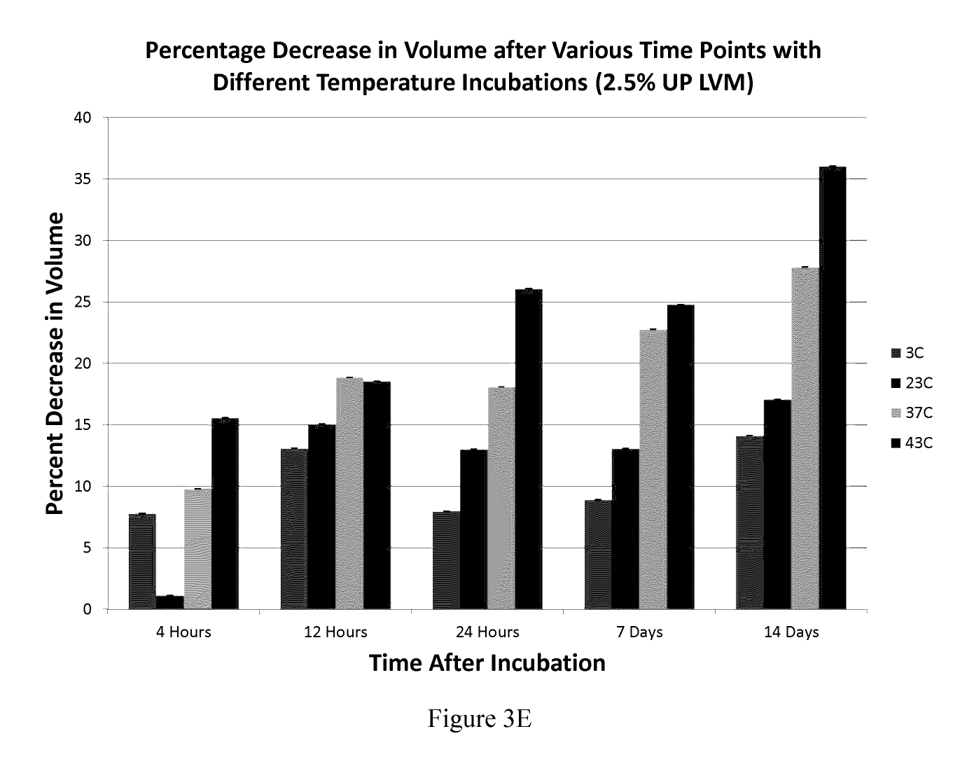

FIGS. 2A-2E and FIGS. 3A-3E demonstrate the effect of in vitro incubation at various temperatures on alginate microcapsule. For example, 0.5% UP LVM microcapsules incubated at 3° C. shows minimal change in size compared to capsules incubated at 37°±5° C. which show a nearly 8% reduction in diameter (see FIG. 2A) and a 25% reduction in volume (see FIG. 3C). FIG. 2B illustrates that the results were reproducible with 2.5% UP LVM capsules containing porcine islets. Similar results have been obtained with ultra-pure low viscosity D-guluronate (“UP LVG”) microcapsules.

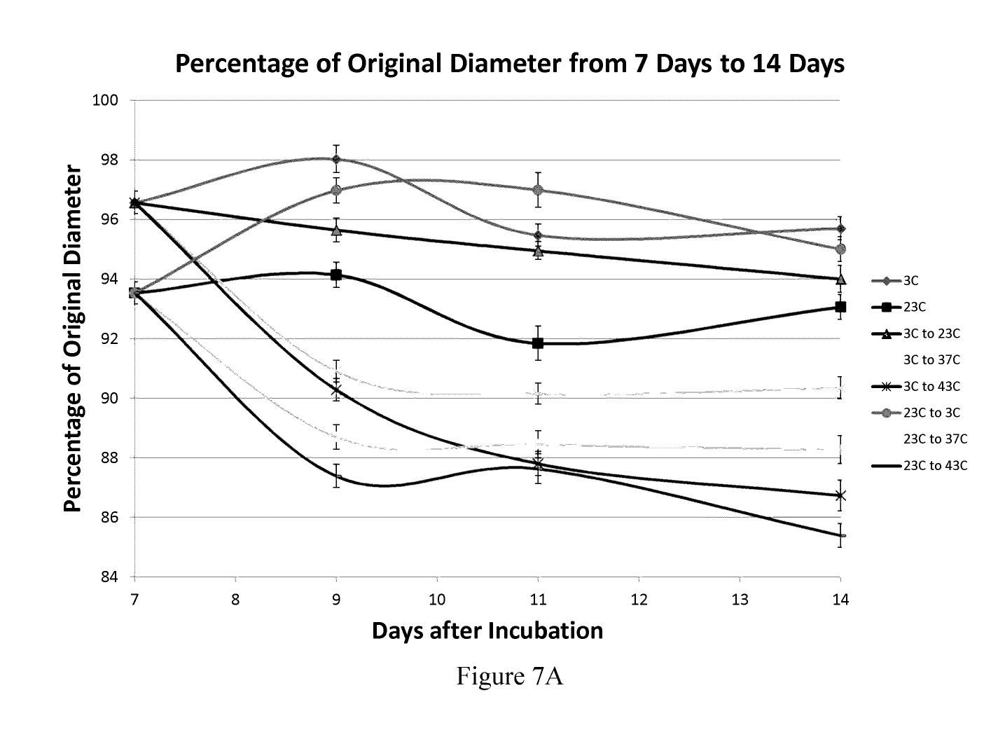

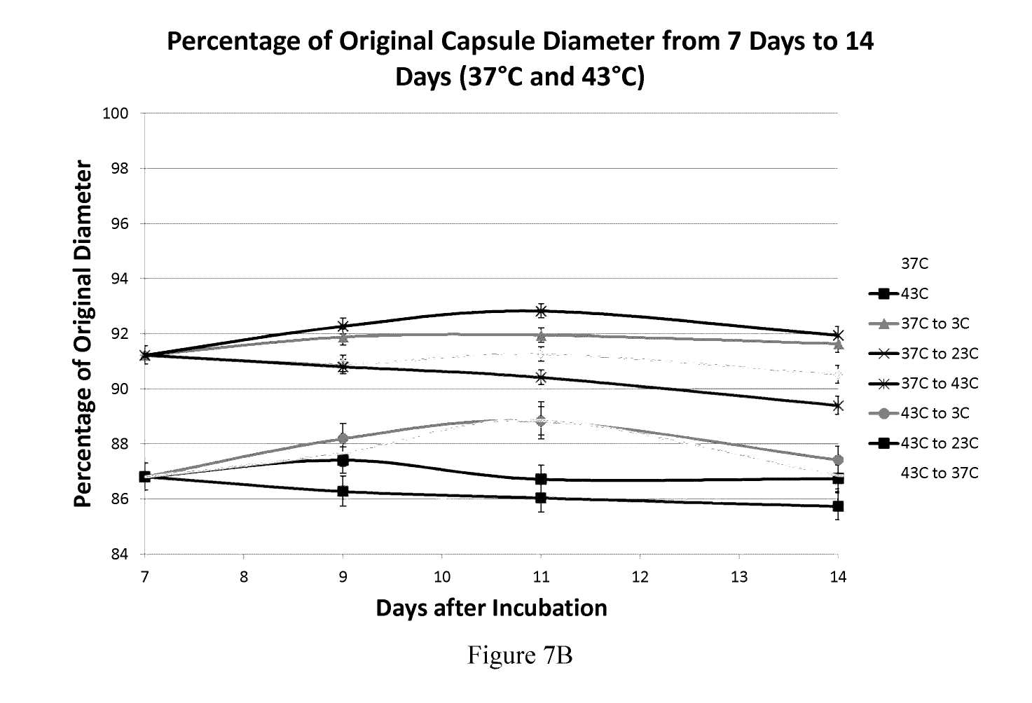

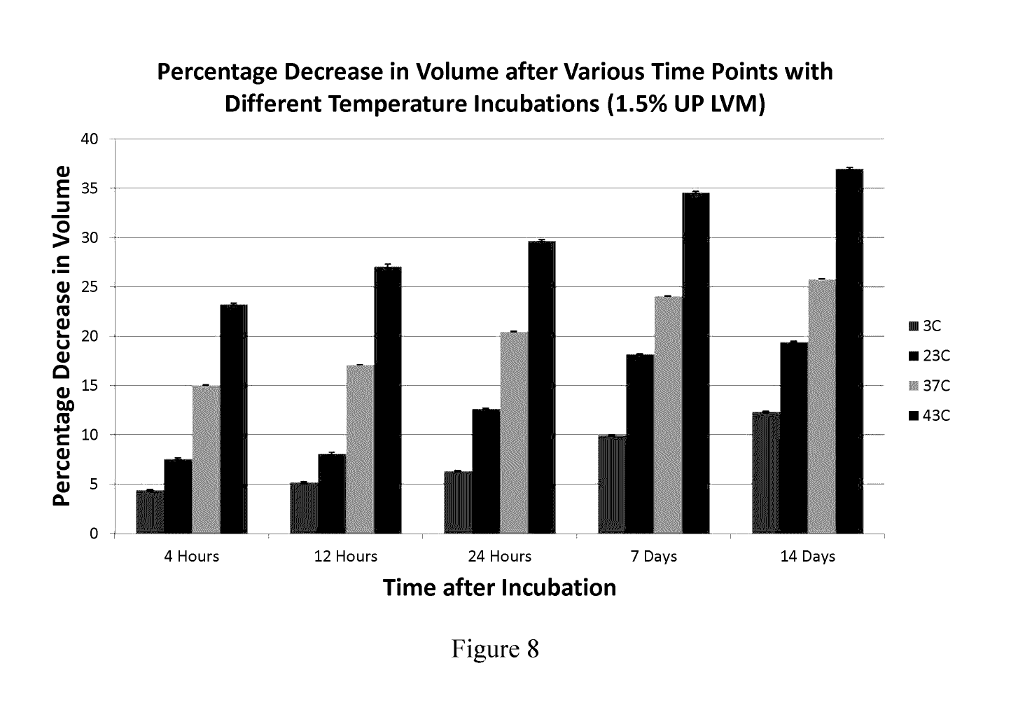

FIGS. 7A-7B and FIG. 8 demonstrate a similar effect of in vitro incubation at various temperatures on alginate microcapsule using 1.5% UP LVM blank capsules. As shown in FIG. 7A, 1.5% UP LVM blank capsules incubated at 3° C. show minimal change in size compared to capsules incubated at 23° C. As will be discussed in more detail below, when microcapsules previously incubated at lower temperatures were transferred to physiological temperatures for an additional 7 days, they demonstrated significant isotropic shrinkage (see FIG. 7A). However as illustrated in FIG. 7B, when the reverse was attempted, there was no significant change in microcapsule size.

FIG. 3E illustrates a general trend of a significant decrease in volume of 2.5% UP LVM microcapsules incubated at physiological body temperatures when compared to lower temperatures. A similar trend is seen in FIG. 8 which shows a general trend of a significant decrease in volume of 1.5% UP LVM microcapsules incubated at physiological body temperatures when compared to lower temperatures.

### Pore Size Measurements

Alginate microcapsule pore size measurements are made using fluorescently tagged 150 kda dextran molecules. Dextrans of this size were chosen to mimic immunoglobulin g (IgG) which can attach to transplanted cells and tissue and trigger an immune attack. We noted that alginate microcapsules incubated at 37°±5° C. showed a significant reduction in dextran diffusion when compared to capsules stored at 3° C. or 23° C.

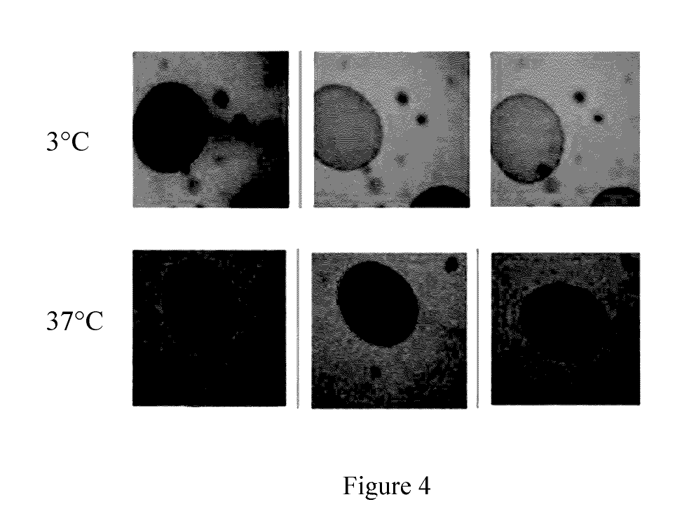

FIG. 4 illustrates dextran permeability using green fluorescent 150 kDa dextrans. As can be seen, the dextran is freely permeable within 2.5% UP LVM Microcapsules at 3° C. but is unable to permeate the same capsules incubated at 37°±5° C. Similar results have been obtained with ultra-pure low viscosity D-guluronate (“UP LVG”) microcapsules.

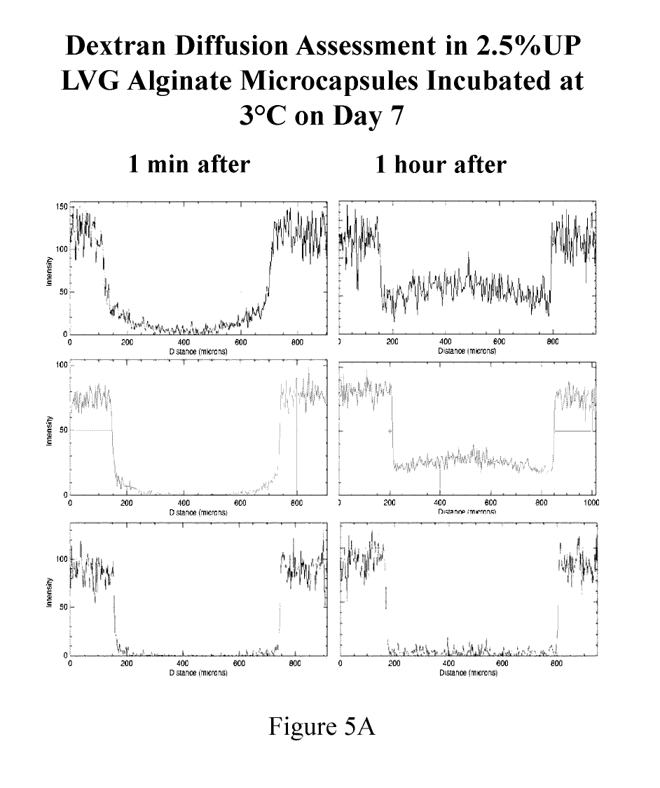

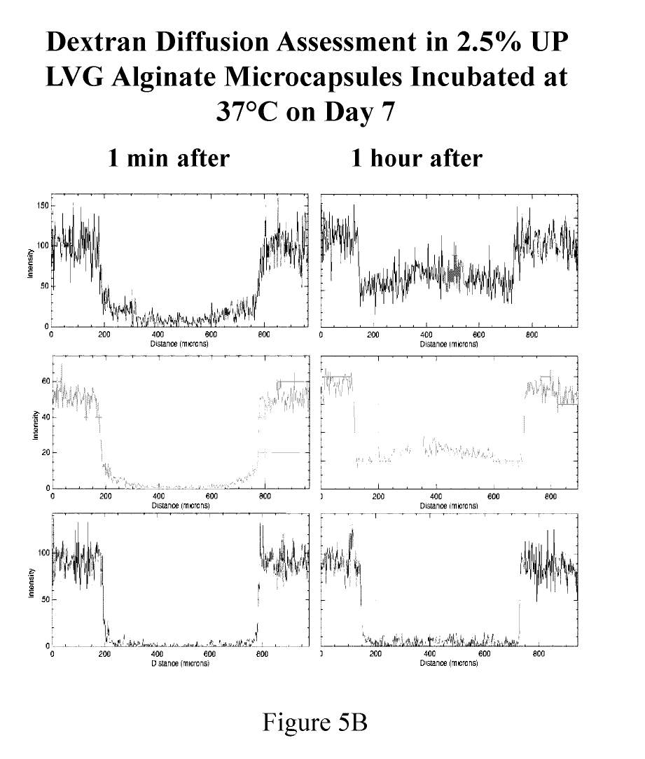

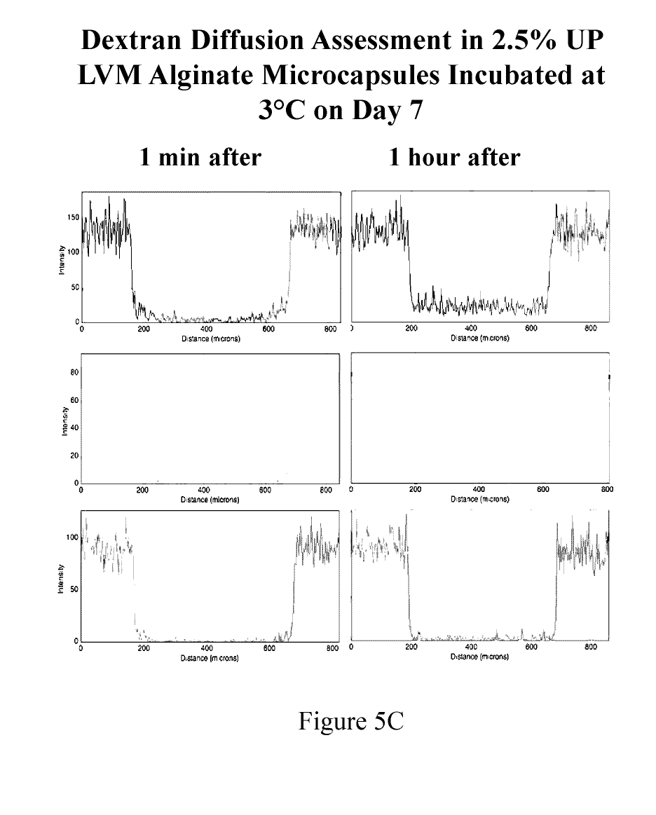

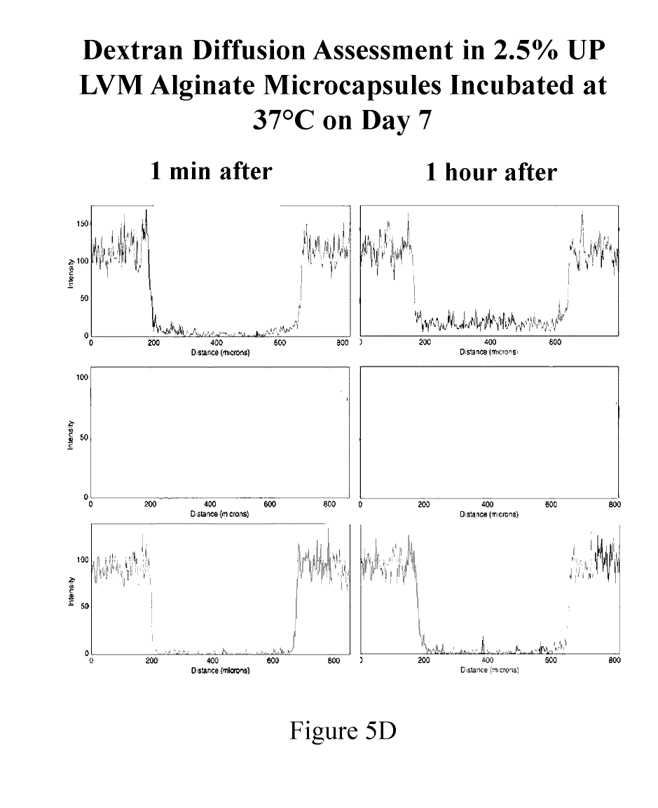

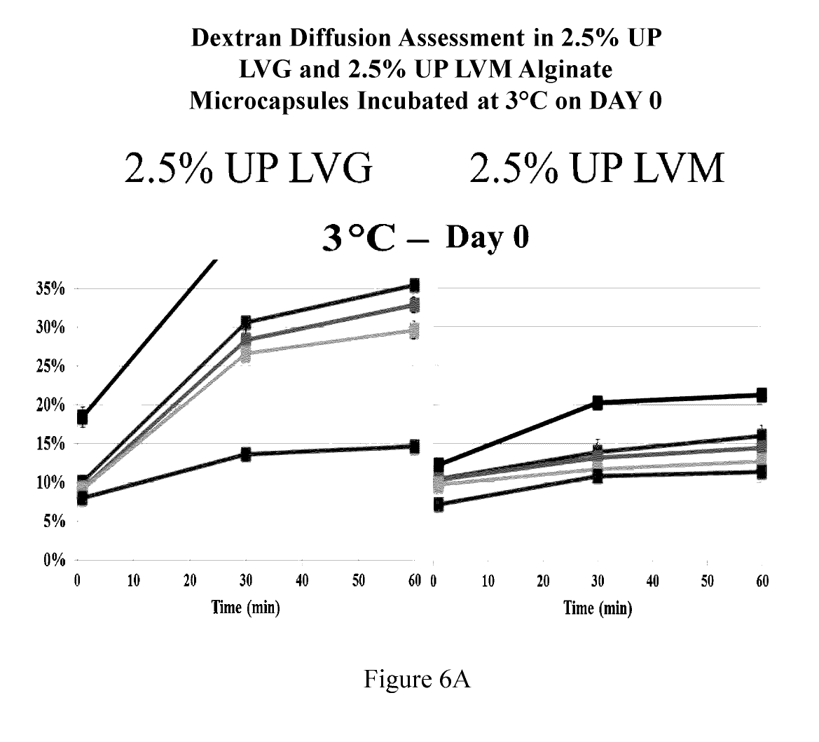

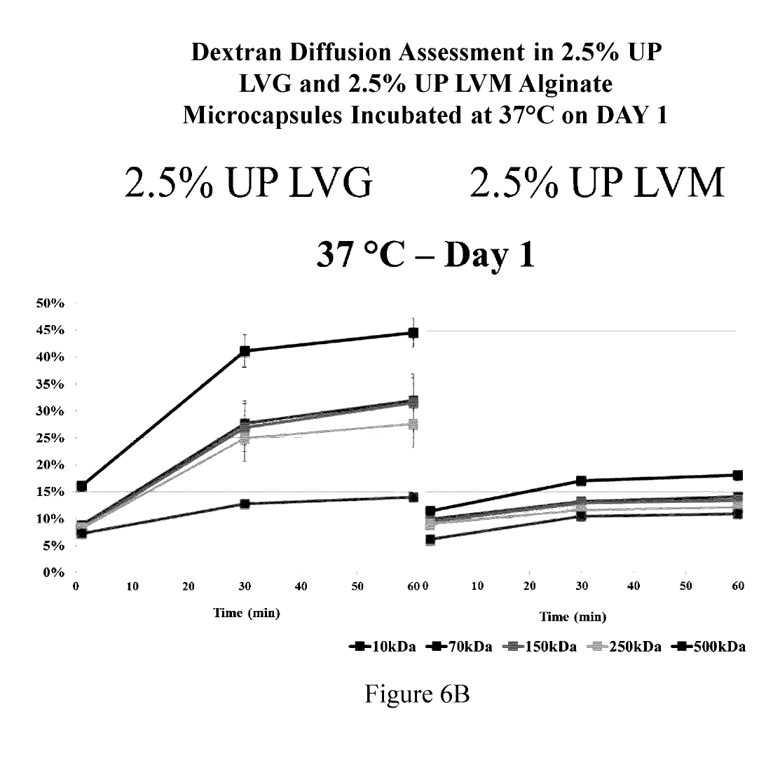

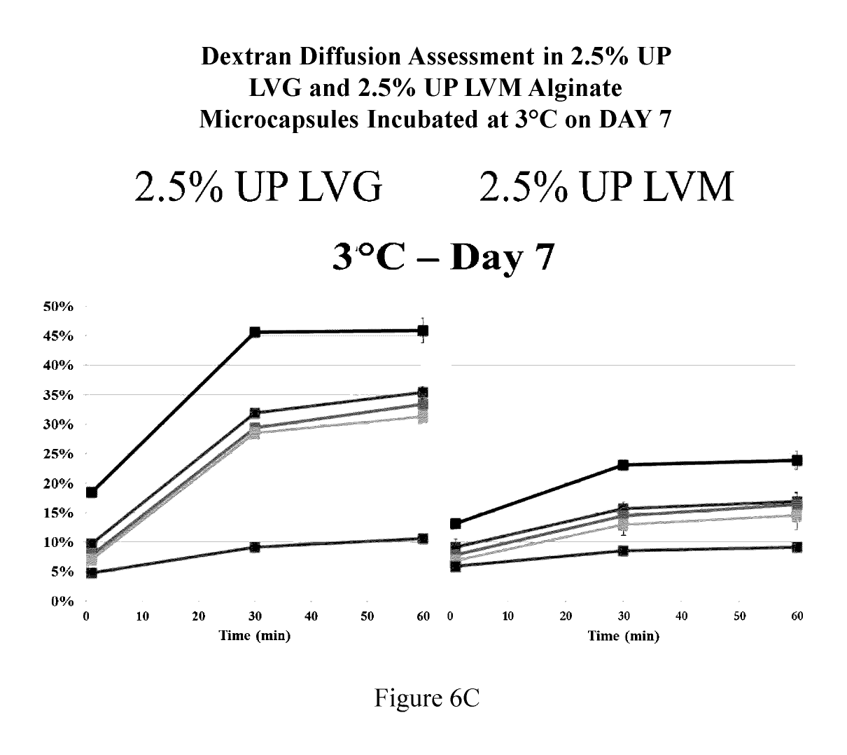

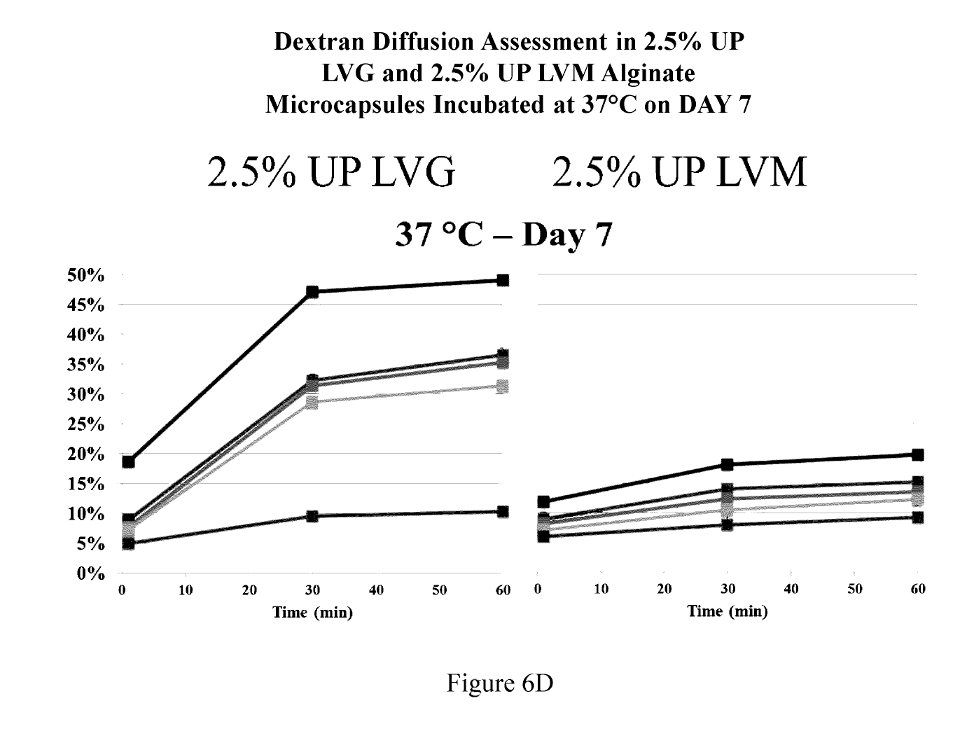

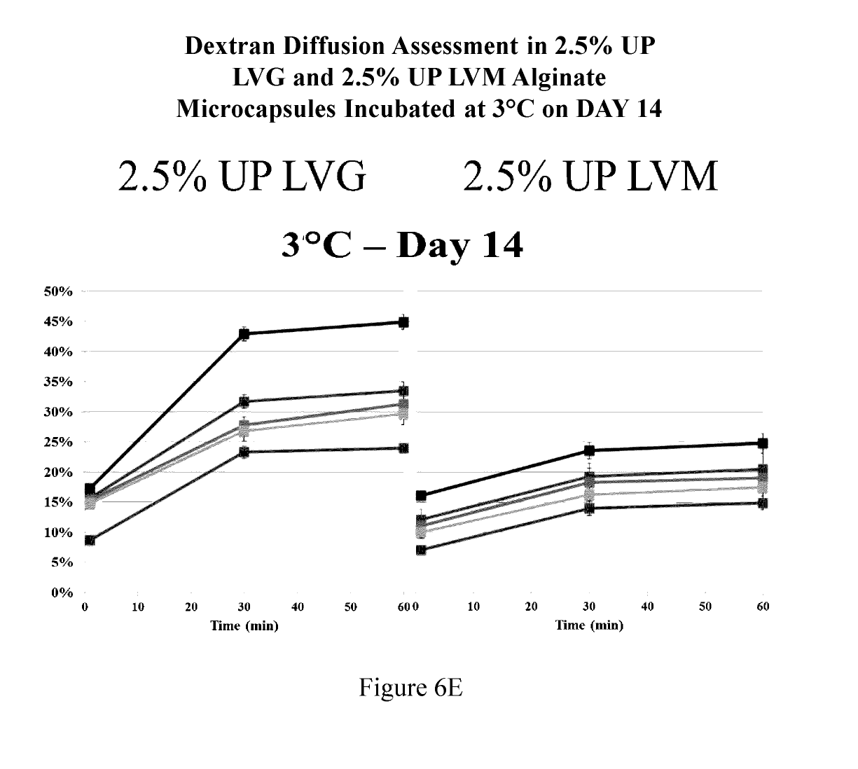

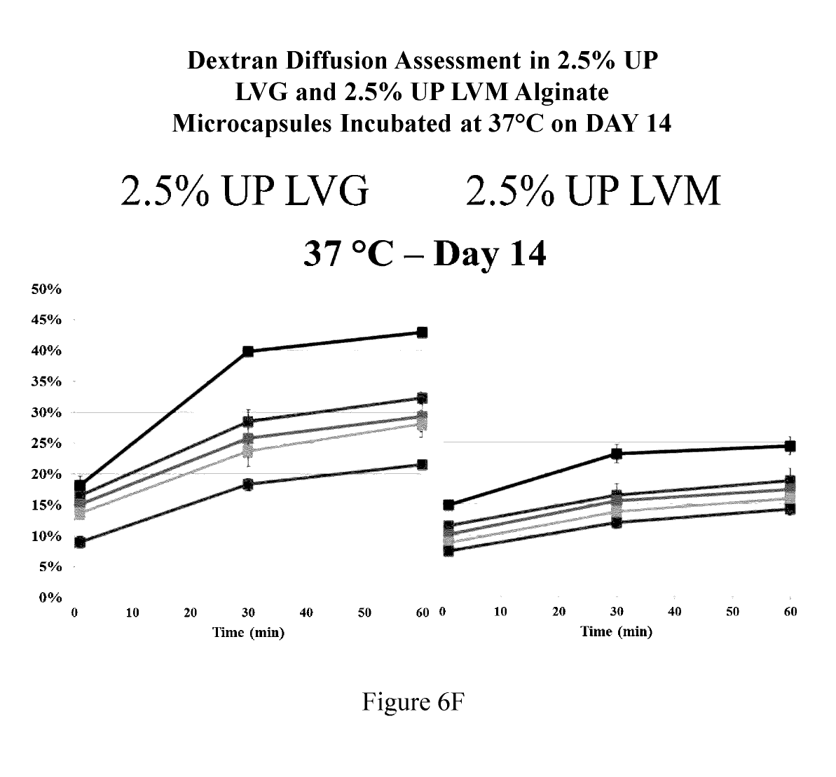

As will be discussed in more detail below, FIGS. 5A-5D and 6A-6D illustrate the dextran diffusion assessment in UP LVM and UP LVG Alginate Microcapsules incubated at different temperatures. As shown, UP LVM alginate microcapsules are relatively impermeable to dextrans of all sizes regardless of dextran size and incubation temperature.

### EXAMPLE 1

As discussed above, islet encapsulation within alginate hydrogels can be used to prevent direct contact between the encapsulated islets and the host immune system while significantly reducing the need for chronic systemic immunosuppression.

In the below disclosed example, alginate microcapsules were incubated at predetermined temperatures to evaluate changes in morphology and permselectivity in order to determine optimal culture conditions for utilization in islet transplantation. Materials and Methods.

Alginate preparation: 2.5% (w/v) alginate solutions made with Ultra Pure Low Viscosity Mannuronate (NovaMatrix® PRONOVA™ UP LVM) and Ultra Pure Low Viscosity Guluronate (NovaMatrix® PRONOVA™ UP LVG) were filtered using a polyethersulfone 32 mm 0.8/0.2 μm filter (PALL Acrodisc® PF) in a Class II biosafety hood.

Microcapsule generation: Alginate microcapsules were generated using an air-pressure-driven electrostatic encapsulator (Nisco Engineering AG) at standard settings (needle height: 30 mm, gelling solution: 120 mM CaCl₂, agitator speed: 80 rpm, air pressure: 3 psi, and voltage: 9 kV. Microcapsules were stored at 3° C., 23° C. or 37° C. in 5 mM CaCl₂.

Image analysis: After 7 days of in vitro culture at these temperatures, the microcapsules were analyzed for changes in their morphology and then transferred to different temperature conditions for an additional 7 days. At 12 hours, 24 hours, 3 days, 7 days, 10 days and 14 days, a minimum of 100 microcapsules were imaged under a phase contrast microscope for changes in morphology. The images were analyzed using the image analysis software, ImageJ, to determine the percentage change in microcapsule size over time.

Dextran diffusion assay: A cocktail of three fluorescently-tagged dextrans were plated in a 12-well plate with the capsules. Cascade blue-conjugated 10 kDa dextran, fluorescein isothyocyanate conjugated 150 kDa dextran, and tetramethylrhodamine isothiocyanate-conjugated 500 kDa dextran were used. Images were obtained using a two-photon confocal microscope (Leica SP8 confocal microscope) at 1 minute, 30 minutes and 60 minutes after plating. The images were analyzed to evaluate changes in dextran diffusion using a graphic user interface (GUI) developed using MATLAB® specifically for the purpose of quickly and accurately evaluating dextran diffusion within alginate microcapsules.

Statistical Analysis: A one way ANOVA followed by a post-hoc Tukey HSD test was used to determine statistical significance with a p<0.05 considered statistically significant.

### Results

Both UP LVM and UP LVG alginate microcapsules exhibited temperature-dependent, isotropic shrinkage over the study period. When compared to pre-incubation measurements (423.8±2.5 μm), microcapsules incubated for 14 days at physiological temperatures demonstrated significantly greater isotropic shrinkage than those incubated at lower temperatures (402.8±2.2 μm; 3° C., 398.1±1.5 μm; 23° C., 380.1±2.2 μm; 37° C., p<0.01).

When microcapsules previously incubated at lower temperatures were transferred to physiological temperatures for an additional 7 days, as expected, they demonstrated significant isotropic shrinkage (410.8±2.0 μm→378.5±1.6 μm; 3° C.→37° C., 404.5±2.2 μm→375.3±1.9 μm; 23° C.→37° C., p<0.05). However, when the reverse was attempted, there was no significant change in microcapsule size (380.1±1.6 μm→378.5±1.6 μm; 37° C.→3° C., 380.1±1.6 μm→375.3±1.9 μm; 37° C.→23° C., p=0.09).

FIGS. 3A, 3B, and 3E illustrate the effect of incubation at various temperatures on alginate microcapsules. As shown, alginate microcapsules demonstrate diameter and volume loss (M>G) when transferred from lower incubation temperatures to higher temperatures. In some embodiments, the diameter and volume loss is irreversible.

FIGS. 5A-5D and 6A-6D illustrate the dextran diffusion assessment in UP LVM and UP LVG Alginate Microcapsules incubated at different temperatures. As shown, UP LVM alginate microcapsules are relatively impermeable to dextrans of all sizes regardless of dextran size and incubation temperature.

### Discussion

As illustrated above, the ability to optimize alginate microcapsule morphology can be an important step in the development of clinical-grade encapsulation strategies for the treatment of diabetes. Isotropic shrinkage noted at physiological temperatures can affect microcapsule permeability, which has profound implications for encapsulated islet survival after transplantation.

### EXAMPLE 2

In the below disclosed study, alginate microcapsules are cultured at predetermined temperatures to evaluate changes in morphology and volume in order to determine optimal culture conditions for utilization in islet transplantation. Additionally, the effect of changes in incubation temperature on alginate microcapsules was evaluated by using dextrans of various molecular sizes to identify capsules with optimal diffusion parameters.

### Materials and Methods

Effect of Temperature on Microcapsule Morphology: Alginate microcapsules were synthesized from 2.5% (w/v) Ultra-Pure Low Viscosity Mannuronate (UP LVM) alginate or 2.5% (w/v) Ultra-Pure Low Viscosity Guluronate (UP LVG, NovaMatrix® PRONOVA™) alginate using an air-driven electrostatic generator (Nisco Engineering AG) at standard settings (Voltage: 9 kV, Agitator Speed: 80 rpm, Pressure: 3 psi, Needle gauge: 25G, Needle height: 25 mm, Gelling solution: 120 mM Calcium Chloride). After encapsulation, the microcapsules were transferred to a 5 mM calcium chloride solution, analyzed for morphology and then stored for a 7-day period at the following temperatures: 3° C., 23° C. and 37° C. After 7 days of in vitro culture at these temperatures, the microcapsules were analyzed for changes in their morphology and then transferred to different temperature conditions for an additional 7 days. At 12 hours, 24 hours, 3 days, 7 days, 10 days and 14 days, a minimum of 100 microcapsules were imaged under a phase contrast microscope for changes in morphology.

Effect of Temperature on Microcapsule Permeability: Blank and porcine islet-containing microcapsules were made with 1.5% (w/v), 2.5% (w/v), and 3.5% (w/v) ultra-pure low viscosity high mannuronate (UP LVM, NovaMatrix® PRONOVA™, Cat. 4200206, Lot. BP-0711-02) and ultra-pure low viscosity high guluronate (UP LVG, NovaMatrix® PRONOVA™, Cat. 4200006, Lot. BP-0710-04) using a microcapsule generator (Nisco Engineering AG, Zürich Switzerland) at standard settings (9 kV; voltage, 80 rpm; stirrer speed, 3±10 psi; air pressure, 25±15 mm; needle height, 25G; needle gauge, 120 mM CaCl₂; gelling solution). Microcapsules were either incubated at 24° C. or 37° C. in either 5 mM CaCl₂ or juvenile porcine islet (JPI) media (Optatio LLC, Irvine, Calif.) for 24-hours before plating the dextran diffusion assay.

A cocktail of three fluorescently-tagged dextrans was added to all wells in a 24-well plate along with 500 μL of either JPI or 5 mM CaCl₂ to match the storage media of the capsule tested. Dextran solutions were plated as follows: 10 μL (7 mg/mL) of cascade blue labeled 10 kDa dextran (Life Technologies, Cat. D-1976, Grand Island, N.Y.), 10 μL (7 mg/mL) of tetramethylrhodamine isothiocyanate (TRITC) labeled 500 kDa dextran (Sigma-Aldrich, Cat. 52194, St. Louis, Mo.), and 10 μL (7 mg/mL) of 70 kDa, 150 kDa, or 250 kDa fluorescein isothyocyanate (FITC) labeled dextran (Sigma-Aldrich, Cat. 90718, FD150S, FD250S, St. Louis, Mo.). Capsule samples were taken post-incubation and 500 μL suspensions were added to each well. Images were obtained (n=10) using a Leica TCS SP8 confocal microscope at 1, 30, and 60 minutes. Each color channel was run using sequential image recording to avoid fluorescent crosstalk. Microscope settings for each channel were set to maximize the number of gray values in the output signal of the photomultiplier (PMT) and the HyD detector. Leica HyD hybrid detectors were used for the red and green channel. A Leica PMT detector was used for the blue channel.

The images were analyzed using a custom Graphical User Interface (GUI) made in house, which was written in MATLAB™ (v. 2014a, Mathworks, Natick, Mass., USA). The GUI primarily determined the percentage change in fluorescence intensity inside the capsules over time.

Statistical Analysis: The experiments were repeated three times to ensure that the results obtained were consistent. A one way ANOVA followed by a post-hoc Tukey HSD test was used to determine statistical significance with a p<0.05 considered statistically significant. All data was reported as mean±SEM. Statistical analysis was performed using a one way ANOVA with a post hoc Tukey test and p<0.05 was considered statistically significant.

### Results

Effect of Temperature on Microcapsule Morphology: Both UP LVM and UP LVG alginate microcapsules exhibited temperature-dependent, isotropic shrinkage over the study period. FIG. 3D illustrates the effect of temperature on alginate capsule diameter. In some examples, alginate capsules were incubated at 3° C., 23° C., 37° C., or 43° C. for 7 days. The capsules were then transferred to 3° C., 23° C., 37° C., or 43° C. for an additional 7 days. Capsule diameter was thereafter measured at 12 hours, 1 day, 3 days, 7 days, 11 days, and 14 days post capsule production. As shown in FIG. 3D, when compared to pre-incubation measurements (423.8±2.5 μm), microcapsules incubated for 14 days at physiological temperatures demonstrated significantly greater isotropic shrinkage than those incubated at lower temperatures (402.8±2.2 μm; 3° C., 398.1±1.5 μm; 23° C., 380.1±2.2 μm; 37° C., p<0.01).

FIG. 3F illustrates the change in microcapsule volume after incubation at different temperatures. In some examples, alginate microcapsules were made at 23° C. then incubated for 14 days at 3° C., 23° C., 37° C., and 43° C. Capsule diameter was measured at 4 hours, 12 hours, 1 day, 7 days, and 14 days. As illustrated in FIG. 3F, when microcapsules previously incubated at lower temperatures were transferred to physiological temperatures for an additional 7 days, as expected, they demonstrated significant isotropic shrinkage (410.8±2.0 μm→378.5±1.6 μm; 3° C.→37° C., 404.5±2.2 μm→375.3±1.9 μm; 23° C.→37° C., p<0.05). However, when the reverse was attempted, there was no significant change in microcapsule size (380.1±1.6 μm→378.5±1.6 μm; 37° C.→3° C., 380.1±1.6 μm 375.3±1.9 μm; 37° C.→23° C., p=0.09).

Effect of Temperature on Microcapsule Permeability: Dextrans of all sizes were observed to begin diffusing immediately inside the capsules for each group at an increasing rate until reaching equilibrium, approximately one hour after plating (i.e. 1.5% UP LVG islet capsule in JPI at 37° C.; 10 kDa dextran; 1 min: 17±0%, 30 min: 62±1%, 60 min: 62±1%; 70 kDa dextran: 1 min: 15±0%, 30 min: 49±1%, 60 min: 49±1%; 150 kDa dextran; 1 min: 14±0%, 30 min: 42±1%, 60 min: 43±1%; 250 kDa dextran; 1 min: 13±0%, 30 min: 39±1%, 60 min: 41±1%; 500 kDa dextran; 1 min: 8±1%, 30 min: 34±1%, 60 min: 36±0%). As a general trend, 10 kDa dextrans showed the greatest diffusion followed by 70 kDa dextrans, 150 kDa dextrans, 250 kDa dextrans, and 500 kDa dextrans, which demonstrated the least amount of diffusion in all groups, suggesting that alginate microcapsules are relatively impermeable to large protein molecules. Interestingly, the presence of islets in the capsules did not significantly affect diffusivity (i.e. 1.5% UP LVM capsule in JPI at 24° C.; 10 kDa dextran; 60 min; islet: 48±1% vs. blank: 48±0), indicating there were no tissue impurities in the capsule walls from the encapsulation method used.

Dextrans showed decreased diffusion as alginate viscosity increased (i.e. 2.5% UP LVM blank capsule in 5 mM CaCl₂ at 37° C.; 150 kDa dextran; 60 min; 31±0% vs. 14±0% in 3.5% capsules). FIG. 3C illustrates a comparison of UPLVM versus UPLVG at 3° C. and 37° C. In some examples, alginate microcapsules were made using 2.5% UPLVM or 2.5% UPLVG. They were incubated for 7 days at 3° C. or 37° C. After 7 days, the capsules were either kept at their original temperature or switched to 3° C. or 37° C. Capsule diameter were measured at 0 hours, 6 hours, 12 hours, 24 hours, 72 hours, 120 hours, 168 hours, 216 hours, 264 hours, and 360 hours. As illustrated, diffusion was shown to be significantly higher in capsules with high mannuronate content over high guluronate content (i.e. 2.5% UP LVM blank capsules in 5 mM CaCl₂ at 24° C.; 70 kDa dextran; 60 min; 35±0% vs. 66±0% in UP LVG capsules).

Dextran diffusion decreased after exposure to JPI media (i.e. 3.5% UP LVG blank capsule in 5 mM CaCl₂ at 37° C.; 10 kDa dextran; 60 min; 65±0% vs. 38±0% in JPI), likely due to the media's sodium chloride content replacing calcium ions in the capsule walls.

Both capsules in JPI media and 5 mM CaCl₂ showed decreased diffusion when stored at 37° C. compared to 24° C. (i.e. 1.5% UP LVG blank capsule in JPI; 10 kDa dextran; 60 min; 37° C.; 48±0% vs. 61±0% in 24° C. capsules), likely due to capsule shrinkage observed at higher temperatures causing tightening of the capsule walls.

FIGS. 6A-6F illustrate the permeability of alginate microcapsules at varying temperature. In some examples, 2.5% UP LVG or UP LVM microcapsules were incubated for 14 days at 3° C. or 37° C. Dextran diffusion was used to measure the capsule permeability at the different temperatures, to molecules of 10 KDa, 70 KDa, 150 KDa, 250 KDa, and 500 KDa. As noted above, diffusion was shown to be significantly higher in capsules with high mannuronate content over high guluronate content. Similarly, greater diffusion is observed at lower temperatures, likely due to capsule shrinkage observed at higher temperatures causing tightening of the capsule wall.

### Discussion

The alginate capsules exhibited time and temperature-dependent, isotropic shrinkage. By the 7-day time point, the diameter of capsules incubated at all temperatures was significantly different (p-value<0.01) than their original size, which correlated to decreases in volume. Microcapsules incubated at 3° C. displayed isotropic shrinkage when transferred to higher temperatures after 7 days, but those incubated at 37° C. and then transferred to lower temperatures did not show this phenomenon. The ability to optimize alginate microcapsule morphology can be an important step in development of clinical-grade encapsulation strategies for the treatment of diabetes. Isotropic shrinkage noted at physiological temperatures is expected to affect microcapsule permeability, which has profound implications for encapsulated islet survival after transplantation. Assays that can evaluate microcapsule permselectivity were performed to determine the optimal culture conditions to achieve the ideal pore size to maximize transplant success in encapsulated islets.

The results of this study suggest that alginate composition (for example guluronic acid content) and temperature influence permeability. In some examples, high mannuronate alginate microcapsules confer better protection from the humoral immune system. In some examples, higher viscosities confer better protection from the humoral immune system. In some examples, higher temperatures confer better protection from the humoral immune system.

While capsules in the aforementioned study that were stored at 24° C. or in 5 mM CaCl₂, encapsulated islets for transplantation are frequently stored in JPI media at 37° C. to preserve tissue viability prior to transplantation. Therefore, in some examples, encapsulated islets that can provide protection from the humoral immune system can be 3.5% UPLVG microcapsules stored in JPI media at 37° C. and 2.5% UPLVM microcapsules stored in JPI media at 37° C. While in some capsule types high mannuronate content showed better protection from 70 kDa to 250 kDa dextrans (representative of IgG antibodies), 3.5% UP LVG capsules stored in JPI at 37° C. demonstrated the same exclusion of these dextrans as their high mannuronate counterparts while imparting 10 kDa diffusion (representative of insulin), which can be desirable for in vivo islet transplants.

The ability to optimize alginate microcapsule morphology can be beneficial in the development of clinical-grade encapsulation strategies for the treatment of diabetes. In some examples, 2.5% UPLVM microcapsules can be predisposed to isotropic shrinkage that can be exacerbated at greater temperatures. This behavior can alter microcapsule porosity and permeability, which can have implications for encapsulated islet survival after transplantation.

Any structure, feature, or step in any embodiment can be used in place of, or in addition to, any structure, feature, or step in any other embodiment, or omitted. This disclosure contemplates all combinations of features from the various disclosed embodiments. No feature, structure, or step is essential or indispensable.

What is claimed is:

1. A method for reducing the volume of alginate microcapsules, comprising:
incubating the alginate microcapsules at between 32°-42° C. under conditions sufficient to reduce the microcapsule volume.

2. A method for producing microcapsules comprising alginate encapsulated islet or stem cells, the method comprising:
mixing islet or stem cells with 0.5%-5% ultra-pure low viscosity D-mannuronate or ultra-pure low viscosity D-guluronate alginate to form an alginate-islet or stem cell mixture;
dripping the alginate-islet or stem cell mixture into a gelling solution to form alginate microcapsules; and
incubating the microcapsules at 32°-42° C. for a time sufficient to shrink the alginate microcapsules thereby reducing the dead space between the alginate microcapsule and the encapsulated islet or stem cells.

3. The method of claim 2, wherein the gelling solution is 10 mM-150 mM calcium chloride.

4. The method of claim 2, further comprising the step of washing the alginate microcapsules with CaCl₂.

5. The method of claim 2, wherein the alginate microcapsules comprise a diameter which is reduced by 8-10% compared to alginate microcapsules not incubated at 32°-42° C.

6. The method of claim 2, wherein the alginate microcapsules comprise a volume which is decreased by 20-27% compared to alginate microcapsules not incubated at 32°-42° C.

7. The method of claim 2, wherein the alginate microcapsules comprising reduced pore size compared to alginate microcapsules not incubated at 32°-42° C.

8. A method for reducing the volume of alginate microcapsules, comprising:
crosslinking the alginate microcapsules with barium chloride; and
incubating the alginate microcapsules at 32°-42° C. under conditions sufficient to reduce the microcapsule volume.

9. A method for producing microcapsules comprising alginate encapsulated islet or stem cells, the method comprising:
mixing islet or stem cells with 0.5%-5% ultra-pure low viscosity D-mannuronate or ultra-pure low viscosity D-guluronate alginate to form alginate-islet or stem cell mixture;
crosslinking the microcapsules with barium chloride by dripping the alginate-islet or stem cell mixture into a gelling solution composed of barium chloride; and
incubating the microcapsules at 32°-42° C. for a time sufficient to shrink the alginate microcapsules thereby reducing the dead space between the alginate microcapsule and the encapsulated islet or stem cells.

10. The method of claim 9, wherein the gelling solution of barium chloride is composed of between 10-150 mM of barium chloride.

11. The method of claim 9, further comprising the step of washing the microcapsules with BaCl₂.

12. The method of claim 9, wherein the alginate microcapsules comprise a diameter which is reduced by 15% compared to alginate microcapsules not crosslinked with barium chloride.

13. The method of claim 9, wherein the alginate microcapsules comprise a volume which is decreased by 40% compared to alginate microcapsules not crosslinked with barium chloride

14. The method of claim 9, wherein the alginate microcapsules comprise a pore size which is reduced compared to alginate microcapsules not incubated at 32°-42° C.

15. The method of claims 1 and 8, wherein the alginate used is 0.5-5 mL of 0.5%-5% ultra-pure low viscosity D-mannuronate or ultra-pure low viscosity D-guluronate alginate.

16. An apparatus for artificial islet or stem cell implantation for the treatment of diabetes, comprising:
an alginate microcapsule comprising an alginate-islet or stem cell mixture,
wherein the alginate is made from 0.5%-5% ultra-pure low viscosity D-mannuronate or ultra-pure low viscosity D-guluronate alginate
wherein the alginate-islet or stem cell mixture is treated with calcium chloride, and
wherein the alginate microcapsule is incubated at 32°-42° C.

17. An apparatus of claim 16, wherein the alginate microcapsules comprise a diameter which is reduced by 8-10% compared to alginate microcapsules not incubated at 32°-42° C.

18. An apparatus of claim 16, wherein the alginate microcapsules comprise a diameter which is reduced by 20-27% compared to alginate microcapsules not incubated at 32°-42° C.

19. An apparatus for artificial islet or stem cell implantation for the treatment of diabetes, comprising:
an alginate microcapsule comprising an alginate-islet or stem cell mixture,
wherein the alginate is made from 0.5%-5% ultra-pure low viscosity D-mannuronate or ultra-pure low viscosity D-guluronate alginate
wherein the alginate-islet or stem cell is treated with barium chloride, and
wherein the alginate microcapsule is incubated at 32°-42° C.

20. An apparatus of claim 19, wherein the alginate microcapsules comprise a diameter which is reduced by 15% compared to alginate microcapsules not crosslinked with barium chloride.

21. An apparatus of claim 19, wherein the alginate microcapsules comprise a diameter which is reduced by 40% compared to alginate microcapsules not crosslinked with barium chloride.
